(19) United States
(12) Patent Application Publication
(10) Pub. No.: US 2012/0322759 A1
(43) Pub. Date: Dec. 20, 2012

(54) SEMULOPARIN FOR THE PREVENTION OF VENOUS THROMBOEMBOLISM IN CANCER PATIENTS RECEIVING CHEMOTHERAPY

(75) Inventors: Umesh CHAUDHARI, Kendall Park, NJ (US); Dominique DESTREE, Paris (FR); Francesca LAWSON, Penn Valley, PA (US); Guillaume LECORPS, Massy (FR)

(73) Assignee: AVENTIS PHARMA SA, Antony (FR)

(21) Appl. No.: 13/469,773

(22) Filed: May 11, 2012

(30) Foreign Application Priority Data
May 12, 2011 (EP) 11305577.6
Jul. 28, 2011 (EP) 11305981.0
Dec. 7, 2011 (EP) 11306623.7
Apr. 13, 2012 (EP) 12305435.5

(51) Int. Cl. A61K 31/727; G06Q 50/22; A61P 7/02

(52) U.S. Cl. 514/56; 705/2

(57) ABSTRACT

The invention relates to an ultra-low molecular weight heparin with an average molecular weight of 2000 to 3000 Daltons, an anti-FXa activity of about 160 U/mg and an anti-FIIa activity of about 2 U/mg, in particular semuloparin, for use as an antithrombotic agent for the prophylaxis of venous thromboembolism in cancer patients receiving chemotherapy for locally advanced or metastatic solid tumors, more specifically in patients receiving chemotherapy for locally advanced or metastatic pancreatic or lung cancer, or for locally advanced or metastatic solid tumors with a VTE risk score equal to or greater than 3.

The invention relates to the use of semuloparin or a pharmaceutically acceptable salt thereof for the prevention of venous thromboembolism in cancer patients receiving chemotherapy.

Semuloparin, or AVE5026 (sanofi-aventis laboratory code), belongs to a new generation of hemisynthetic heparins. It is a new ultra-low molecular weight heparin, with an average molecular weight of 2000-3000 Daltons and a novel antithrombotic profile resulting from high anti-Factor Xa activity (˜160 U/mg) and residual anti-Factor IIa activity (˜2 U/mg). It is obtained by selective and controlled depolymerization of heparin by a phosphazene base, as described for example in Journal of Thrombosis and Haemostasis, 2009, vol. 7, 1143-1151. Semuloparin, in the form of its sodium salt, is in clinical development for venous thromboembolism prevention.

Venous thromboembolism (VTE), including pulmonary embolism (blood clots flowing to the lungs, which may be life-threatening or fatal), is a complication of concern for cancer patients. Some studies have highlighted a 4 to 7 fold higher risk for VTE in patients suffering from cancer, compared to patients with other diseases (Falanga A., Cancer Invest., 2009, 27, 105-115).

This higher VTE risk results from multiple mechanisms including direct interaction by the tumor cells with the hemostatic system and activation of the coagulation cascade, abnormal hemodynamics in cancer patients, as well as from prothrombotic properties of anticancer agents. In fact, chemotherapy can induce direct vascular damage and release of procoagulants and cytokines from damaged tumor cells, further increasing the incidence of VTE (Haddad T. et al., Thromb. Res., 2006, 118, 555-568).

In fact, VTE is one of the leading causes of death in cancer patients (Khorana A. A. et al., J. Thromb. Haemost., 2007, 5, 632-634). This pathology has been described as being a significant predictor of 1 year mortality in a variety of cancers including lung, colon/rectum, melanoma, breast, uterus, ovarian, prostate and non-Hodgkin lymphoma (Chew H. K. et al., Arch. Intern. Med., 2006, 166, 458-464). VTE is also associated with increased morbidity including recurrent VTE and bleeding complications related to the high doses of anticoagulant required for treatment of VTE (Prandoni P. et al., Blood, 2002, 100, 3484-3488).

Several international oncologic and thrombosis groups have developed guidelines for the prevention of VTE in cancer patients (see for example ACCP guidelines, Geerts W. H. et al. in Chest, 2008, 133, 381S-453S; ASCO guidelines, Lyman G. et al. in Journal of Clinical Oncology, 2007, 25, no. 34, 5490-5505; ESMO guidelines, Mandala M. et al. in Annals of Oncology, 2010, 21, suppl. 5, v274-v276). All of them recommend VTE prophylaxis in hospitalized cancer patients and in cancer patients undergoing surgery, however the recommendation of the guidelines is against routine use of thromboprophylaxis for the primary prevention of VTE in ambulatory cancer patients.

For ambulatory cancer patients receiving chemotherapy, the guidelines either consider or recommend thromboprophylaxis only for selected patients receiving highly thrombogenic antiangiogenic therapy (patients, especially myeloma patients, receiving thalidomide or lenalidomide associated with dexamethasone or chemotherapy). Recently revised oncology guidelines have been updated to include “consideration” of VTE prophylaxis in outpatients based on the baseline assessment of their VTE risk (NCCN Clinical Practice Guidelines in Oncology: Venous Thromboembolic Disease, V.2.2011—ESMO Clinical Practice Guidelines in Annals of Oncology, 2011, vol. 22, suppl. 6, vi85-vi92), stressing nevertheless the need for prospective randomized data to assess the clinical benefit of routine VTE prophylaxis in cancer outpatients.

The guidelines do not recommend routine thromboprophylaxis for ambulatory cancer patients receiving chemotherapy due to lack of conclusive randomized clinical trials demonstrating the favorable benefit-to-risk ratio of anticoagulation in this setting. Indeed, a recent meta-analysis of seven VTE prevention trials with Low Molecular Weight Heparins (LMWHs) in cancer outpatients indicated that thromboprophylaxis significantly reduced the incidence of VTE, but was associated with an 85% increase in major bleeding (Kuderer N. M. et al., Journal of Clinical Oncology 27:15s, 2009 (suppl; abstr 9537)). One of the studies included in the meta-analysis was the PROTECHT study that investigated the LMWH nadroparin versus placebo for tromboprophylaxis in patients with locally advanced or metastatic solid cancer receiving chemotherapy: the incidence of thromboembolic events (using as primary efficacy outcome the composite of symptomatic deep-vein thrombosis of lower or upper limbs, pulmonary embolism, visceral or cerebral venous thrombosis, acute myocardial infarction, ischaemic stroke, acute peripheral arterial thromboembolism, and unexplained death of possible thromboembolic origin occurring during the study treatment plus 10 days) was 3.9% in the placebo group and 2.0% in the nadroparin group, but 5 patients (0.7%) suffered a major bleeding in the nadroparin group while no placebo-treated patients experienced a major bleeding (G. Agnelli et al., Lancet Oncol 2009; 10:943-49).

There is currently no registered treatment indicated for the primary prevention of VTE in cancer patients receiving chemotherapy.

Enoxaparin has been tested in a small number of patients with pancreatic cancer for its use in thromboprophylaxis (U. Pelzer et al., European Journal of Cancer, Supplement, 2009, vol. 7, No. 2, p. 365). The results of this trial have however never been confirmed in a larger trial, and in any case cannot be transposed to patients having other types of solid cancers. Concerning data on bleeding events in this trial, they suffer from insufficient statistical significance. Moreover, enoxaparin was administered at a high, curative dose for the first three months of the trial (1 mg/kg of body weight), therefore data are missing to assess efficacy and safety results with a prophylactic dose of enoxaparin.

Dalteparin has also been tested in a phase IIb trial in patients with pancreatic cancer (A. Maraveyas et al. in European Journal of Cancer Supplements, September 2009, vol. 7, No. 2, page 362 and in Thrombosis research, 2010, vol. 125, suppl. 2, S161). A significant reduction of overall VTE is observed, however here again the antithrombotic product was administered at a therapeutic dose (200 U/kg one a day for the first month and then 80% of the initial dose for 5 months) and data to assess its clinical benefit, including safety parameters, are missing.

Targeting a different patient population (cancer patients with newly diagnosed, objectively confirmed deep vein thrombosis and/or pulmonary embolism), the LMWH dalteparin is indicated for the extended treatment of symptomatic VTE to reduce the recurrence of such events in patients with cancer. Enoxaparin has also been studied in a similar group of patients (cancer patients with acute symptomatic VTE) for the prevention of recurrences of VTE after a 6 month-administration (Deitcher S. R. et al., Clin. Appl. Thromb. Hemost., 2006, 12, 389-396).

In still another category of patients (patients undergoing cancer surgery), enoxaparin has been tested for the prophylaxis against VTE when administered for four weeks after surgery for abdominal or pelvic cancer (Bergvist D. et al., N. Engl. J. Med., 2002, vol. 346, No. 13, 975-980).

There is therefore a critical unmet medical need for the prevention of VTE in the specific population of cancer patients who are receiving chemotherapy and who are therefore at risk for VTE, but no antithrombotic drug so far has been demonstrated to be effective and safe for such a category of patients.

The Applicant has now demonstrated, based on a large phase III trial, that a product outside of the LMWH class, namely the Ultra-Low Molecular Weight Heparin (ULMWH) semuloparin, is effective and safe for the prevention of VTE and VTE-related death in cancer patients receiving chemotherapy.

Therefore, the subject-matter of the invention is an ultra-low molecular weight heparin (ULMWH) with an average molecular weight of 2000 to 3000 Daltons, an anti-Factor Xa (anti-FXa) activity of about 160 U/mg and an anti-Factor IIa (anti-FIIa) activity of about 2 U/mg, for use as a safe and effective antithrombotic agent for the prophylaxis of venous thromboembolism (VTE) in cancer patients receiving chemotherapy for locally advanced or metastatic solid tumors.

The anti-FXa and anti-FIIa activities described above are measured using amidolytic methods on a chromogenic substrate as adapted from the monograph on LMWHs of the European Pharmacopeia in force, using as reconstitution buffer a tris-NaCl pH 7.4 buffer comprising PEG6000 (polyethylene glycol 6000) instead of albumin, and an ULMWH reference substance with an anti-FXa activity of 159 U/mg and an anti-FIIa activity of 2.9 U/mg. The potencies are expressed in units per mg due to the use of an internal ULMWH reference standard. Indeed, as a function of the concentration/dilution, lack of parallelism can be observed for anti-FIIa activities routine determination when the ULMWH is calibrated versus LMWH standard. The anti-FXa and anti-FIIa activities of the ULMWH reference substance described above have been determined relative to the international LMWH standard on a range of dilution where the parallelism was obtained. The results of the dosages are exploited according to §5.3 of the European Pharmacopeia in force (“Statistical analyses of dosages and biological assays results”).

More particularly, the above ULMWH is semuloparin and the subject-matter of the invention is therefore semuloparin for use as a safe and effective antithrombotic agent for the prophylaxis of VTE in cancer patients receiving chemotherapy for locally advanced or metastatic solid tumors.

The term “semuloparin”, in the framework of the instant invention, encompasses any pharmaceutically acceptable salt thereof, in particular its sodium salt. The term “semuloparin” shall therefore be understood herein as “semuloparin or any pharmaceutically acceptable salt thereof”.

According to the invention, “cancer patients” are defined as patients having cancer and being under chemotherapy treatment. In the framework of the invention, said “cancer patients” may be ambulatory patients (outpatients) or hospitalized patients.

In an embodiment of the invention, the cancer patients exclude those with severely restricted mobility, i.e. bedridden patients.

In another embodiment, the cancer patients in the framework of the invention are ambulatory patients (i.e. excluding patients hospitalized for cancer surgery and medical patients with severely restricted mobility).

In still another embodiment, the cancer patients in the framework of the invention receive chemotherapy without concomitant treatment with thalidomide or lenalidomide.

Concerning the cancer, it can be located for example in the lungs, pancreas, stomach, colon, rectum, bladder or ovaries.

In an embodiment of the invention, the cancer patients receive chemotherapy for locally advanced or metastatic solid tumors of the lungs, stomach, colon, rectum, bladder or ovaries.

In another embodiment of the invention, the cancer patients receive chemotherapy for locally advanced or metastatic solid tumors and are at increased risk of VTE. In the framework of the invention, the following patients are considered being at increased risk of VTE:
patients with pancreatic cancer; and/or
patients with lung cancer; and/or
patients with a VTE risk score equal to or greater than 3.

Said “VTE risk score” has been developed and validated for cancer patients receiving chemotherapy by Khorana A. A. et al. in Blood, 2008, vol. 111, 4902-7.

According to such a risk score, the following patients are assigned a risk score of 3 or higher:
patients with pancreatic or stomach cancer who have one or more of the risk factors below before initiating chemotherapy;
patients with lung, bladder or ovarian cancer who have two or more of the risk factors below before initiating chemotherapy;
patients with colon/rectum cancer who have three or more of the risk factors below before initiating chemotherapy.

Risk Factors:
platelet count of 350×10⁹/L or more;
hemoglobin less than 100 g/L or requiring red cell growth factors;
leukocyte count more than 11×10⁹/L;
BMI (body mass index) of 35 kg/m² or more.

Therefore, the subject-matter of the invention is also an ULMWH, in particular semuloparin, for use as a safe and effective antithrombotic agent for the prophylaxis of VTE in patients receiving chemotherapy for locally advanced or metastatic pancreatic or lung cancer or for locally advanced or metastatic solid tumors with a VTE risk score equal to or greater than 3.

More particularly, the subject-matter of the invention is an ULMWH, in particular semuloparin, for use as a safe and effective antithrombotic agent for the prophylaxis of VTE in patients receiving chemotherapy for locally advanced or metastatic pancreatic cancer.

Moreover, the subject-matter of the invention is an ULMWH, in particular semuloparin, for use as a safe and effective antithrombotic agent for the prophylaxis of VTE in patients receiving chemotherapy for locally advanced or metastatic lung cancer.

Moreover, the subject-matter of the invention is an ULMWH, in particular semuloparin, for use as a safe and effective antithrombotic agent for the prophylaxis of VTE in patients receiving chemotherapy for locally advanced or metastatic solid tumors with a VTE risk score equal to or greater than 3, namely patients receiving chemotherapy for locally advanced or metastatic solid tumors selected from:
patients with stomach cancer who have, before initiating chemotherapy, one or more of the risk factors selected from: platelet count of 350×10⁹/L or more; hemoglobin less than 100 g/L or requiring red cell growth factors; leukocyte count more than 11×10⁹/L; body mass index of 35 kg/m² or more;
patients with bladder or ovarian cancer who have two or more of the risk factors defined above before initiating chemotherapy;
patients with colon and/or rectum cancer who have three or more of the risk factors defined above before initiating chemotherapy.

The invention therefore also concerns an ULMWH, in particular semuloparin, for use as a safe and effective antithrombotic agent for the prophylaxis of VTE in patients receiving chemotherapy for locally advanced or metastatic pancreatic or lung cancer or for locally advanced or metastatic cancers of the stomach, bladder, colon/rectum or ovary with a VTE risk score equal to or greater than 3.

In another embodiment, the subject-matter of the invention is an ULMWH, in particular semuloparin, for use as a safe and effective antithrombotic agent for the prophylaxis of VTE in cancer patients receiving chemotherapy for locally advanced or metastatic solid tumors who are at increased risk of VTE, i.e., patients with pancreatic cancer, patients with lung cancer, and patients with other solid tumors and at least one additional VTE risk factor.

The subject-matter of the invention is also an ULMWH, in particular semuloparin, for use as a safe and effective antithrombotic agent for the prophylaxis of VTE in cancer patients receiving chemotherapy for locally advanced or metastatic solid tumors who are at increased risk of VTE, i.e., patients with pancreatic cancer, patients with lung cancer, and patients with bladder, colon, rectum, ovary or stomach cancer and having at least one additional VTE risk factor.

According to the invention, “prophylaxis” refers to the administration of a therapy to an individual who is considered as being at risk for a thromboembolic pathology such as venous thromboembolism (including deep vein thrombosis, which may lead to pulmonary embolism), i.e. to an individual who does not already have symptoms of an established venous thromboembolic state.

In an embodiment of the instant invention, said ULMWH is used in the above patients for the prophylaxis of VTE and VTE-related death.

In another embodiment, said ULMWH is used in the above patients for the prophylaxis of deep vein thrombosis (DVT).

More particularly, said ULMWH is used in the above patients for the prophylaxis of DVT of the upper limbs, including thrombosis related to central venous catheter (CVC-related thrombosis).

In another embodiment, said ULMWH is used in the above patients for the prophylaxis of DVT of the lower limbs, whether proximal or distal.

In still another embodiment, said ULMWH is used in the above patients for the prophylaxis of pulmonary embolism (PE).

In the framework of present invention, the terms below have the following meanings:
“venous thromboembolism” (VTE): designates the formation of a blood clot (named “thrombus”) inside a blood vessel, obstructing the flow of blood through the circulatory system;
“deep vein thrombosis” (DVT): designates a blood clot in a deep vein, more particularly in the lower limbs (legs veins) or in the upper limbs (arms veins);
“proximal DVT”: designates a DVT event in the lower limbs occurring above the knee;
“distal DVT”: designates a DVT event in the lower limbs occurring below the knee;
“pulmonary embolism” (PE): designates a blockage of the main artery of the lung or one of its branches by a thrombus that has traveled from elsewhere in the body through the bloodstream (embolism), usually from the deep veins in the legs.

In an embodiment of the instant invention, said ULMWH displays a favorable clinical net benefit for its use in the above patient population. The clinical net benefit refers to a favorable benefit-to-risk profile (i.e. an improved benefit-risk assessment compared to placebo, as it will be described in details hereafter), “benefit” referring to a reduction in the occurrence of VTE events and VTE-related death and “risk” referring to the occurrence of bleeding events, more particularly clinically relevant bleedings or major bleedings (as defined hereafter).

According to the instant invention, the efficacy and safety of said ULMWH in the therapeutic uses described above is proven by a phase III clinical trial. A “phase III clinical trial” refers, in the framework of the instant invention, to a multinational, randomized, double-blinded study involving a large patients group (more than 3000, as it will be described in details below), as defined by the health authorities and the regulatory laws and guidelines, aiming at being the definitive assessment of how effective and safe the drug is. According to the instant invention, and as it will be described in details below, said phase III clinical trial is performed in cancer patients, more specifically in cancer patients who are undergoing chemotherapy.

According to the instant invention, the ULMWH is administered at a prophylactic dose, namely at a 20 mg daily dose.

According to the instant invention, the patients to which the ULMWH is administered do not display severe renal impairment, which means that their estimated creatinine clearance (CLcr) value, calculated using the well-known Cockroft-Gault formula, shell not be less than 30 mL/min.

In an embodiment of the instant invention, the ULMWH is administered once daily.

Generally, administration of the ULMWH is started at the initiation of a course of chemotherapy and continued daily throughout any subsequent chemotherapy cycles for 3 months, or longer if the increased risk of VTE persists. In the phase III clinical trial of semuloparin in cancer patients described above, the median duration of treatment was 3.5 months.

In another embodiment of the invention, administration of the ULMWH is advantageously started at the initiation of a course of chemotherapy and continued once daily for 3 months. If chemotherapy is definitely discontinued, then treatment with the ULMWH should also be discontinued.

As used herein, the wording “an ULMWH for use as . . .” shall be understood as being equivalent to the wording “use of an ULMWH for . . .” or “use of an ULMWH for the preparation of a medicament for use in . . .”.

The invention therefore also relates to the use of an ULMWH as defined above for the manufacture of a medicament useful as a safe and effective antithrombotic agent for the prophylaxis of VTE in cancer patients receiving chemotherapy for locally advanced or metastatic solid tumors. All the embodiments and features described above also apply to said use.

The invention also relates to an article of manufacture comprising:
a packaging material,
a compound chosen from an ULMWH with an average molecular weight of 2000 to 3000 Daltons, an anti-FXa activity of about 160 U/mg and an anti-FIIa activity of about 2 U/mg, in particular semuloparin or a pharmaceutically acceptable salt thereof, and
a label or package insert contained within said packaging material indicating that said compound is effective as an antithrombotic agent for the prophylaxis of venous thromboembolism (VTE) in cancer patients receiving chemotherapy for locally advanced or metastatic solid tumors.

The invention also relates to an article of manufacture comprising:
a packaging material,
a compound chosen from an ULMWH with an average molecular weight of 2000 to 3000 Daltons, an anti-FXa activity of about 160 U/mg and an anti-FIIa activity of about 2 U/mg, in particular semuloparin or a pharmaceutically acceptable salt thereof, and
a label or package insert contained within said packaging material indicating that said compound is effective as an antithrombotic agent for the prophylaxis of venous thromboembolism (VTE) in patients receiving chemotherapy for locally advanced or metastatic pancreatic or lung cancer or for locally advanced or metastatic solid tumors with a VTE risk score equal or greater than 3, as defined above.

The invention also relates to an article of manufacture comprising:
a packaging material,
a compound chosen from an ULMWH with an average molecular weight of 2000 to 3000 Daltons, an anti-FXa activity of about 160 U/mg and an anti-FIIa activity of about 2 U/mg, in particular semuloparin or a pharmaceutically acceptable salt thereof, and
a label or package insert contained within said packaging material indicating that said compound is safe as an antithrombotic agent for the prophylaxis of venous thromboembolism (VTE) in cancer patients receiving chemotherapy for locally advanced or metastatic solid tumors.

The invention also relates to an article of manufacture comprising:
a packaging material,
a compound chosen from an ULMWH with an average molecular weight of 2000 to 3000 Daltons, an anti-FXa activity of about 160 U/mg and an anti-FIIa activity of about 2 U/mg, in particular semuloparin or a pharmaceutically acceptable salt thereof, and
a label or package insert contained within said packaging material indicating that said compound is safe as an antithrombotic agent for the prophylaxis of venous thromboembolism (VTE) in patients receiving chemotherapy for locally advanced or metastatic pancreatic or lung cancer or for locally advanced or metastatic solid tumors with a VTE risk score equal or greater than 3, as defined above.

The invention also relates to a method of providing semuloparin or a pharmaceutically acceptable salt thereof, wherein said semuloparin or a pharmaceutically acceptable salt thereof is provided along with information describing that semuloparin or a pharmaceutically acceptable salt thereof is indicated in patients receiving chemotherapy for locally advanced or metastatic pancreatic or lung cancer or for locally advanced or metastatic solid tumors with a VTE risk score equal or greater than 3, as defined above.

The invention also relates to a method of promoting the use of semuloparin or a pharmaceutically acceptable salt thereof, the method comprising the step of conveying to a recipient at least one message chosen from:
semuloparin or a pharmaceutically acceptable salt thereof should be prescribed to a patient receiving chemotherapy for locally advanced or metastatic solid tumors;
semuloparin or a pharmaceutically acceptable salt thereof should be prescribed to a patient receiving chemotherapy for locally advanced or metastatic solid tumors of the lungs, pancreas, stomach, colon, rectum, bladder or ovaries;
semuloparin or a pharmaceutically acceptable salt thereof should be prescribed to a patient receiving chemotherapy for locally advanced or metastatic pancreatic cancer;
semuloparin or a pharmaceutically acceptable salt thereof should be prescribed to a patient receiving chemotherapy for locally advanced or metastatic lung cancer; and
semuloparin or a pharmaceutically acceptable salt thereof should be prescribed to a patient receiving chemotherapy for locally advanced or metastatic solid tumors with a VTE risk score equal or greater than 3, as defined above.

Having now described the present invention, the same will be more clearly understood by reference to the following examples of the invention, which are included herewith for purposes of illustration only and are not intended to be limiting of the invention.

### BRIEF DESCRIPTION OF THE DRAWINGS

The enclosed FIGURE (FIG. 1) represents the survival curves for the primary efficacy endpoint (VTE or VTE-related death during the efficacy analysis period, according to time to first VTE event—Cumulative incidence functions by treatment group—Intent-To-Treat population).

The following abbreviations shall be used:

CI: Confidence Interval

CIAC: Central Independent Adjudication Committee

CIFs: Cumulative Incidence Functions

CVC: central venous catheter

CVL: Central Venous Line

CT: computer tomography

DVT: deep vein thrombosis

HR: Hazard Ratio

IP: investigational product

LMWH: low molecular weight heparin

OR: Odds Ratio

PE: pulmonary embolism

UFH: unfractionated heparin

q.d.: quaque die (once daily)

(S)AEs: (serious) adverse events

s.c.: subcutaneously

vs.: versus

VTE: venous thromboembolism

95% (mid-p) CI: 95% (mid-p) Confidence Interval

The SAVE-ONCO Phase III Study:

A multinational, randomized, double blind, placebo-controlled study to evaluate the efficacy and safety of AVE5026 in the prevention of venous thromboembolism (VTE) in cancer patients at high risk for VTE and who are undergoing chemotherapy (ClinicalTrials.gov number: NCT00694382)

1) Study Objectives The primary objective of the study was to compare the efficacy of once daily (q.d.) subcutaneous (s.c.) injections of 20 mg AVE5026 with placebo in the prevention of VTE in cancer patients at high risk for VTE and who are undergoing chemotherapy.

The secondary objectives of the study comprised the evaluation of the safety of AVE5026 in cancer patients at high risk for VTE and who are undergoing chemotherapy, to document AVE5026 exposures and to assess the survival status at one year in this population.

2) Study Design

This was a multinational, multicenter, randomized, double-blind superiority study, with two parallel groups study.

The study was placebo-controlled, as a systematic venous thromboprophylaxis is neither recommended nor routinely used in patients undergoing chemotherapy and as there is no anticoagulant drug approved to date in this indication.

Cancer patients at high VTE risk, defined as patients with a metastatic or locally-advanced solid tumor of the lung, pancreas, stomach, colon/rectum, bladder or ovary and who are undergoing chemotherapy, were randomly assigned to receive once daily s.c. injection of either AVE5026 or placebo:

(i) until change in the initial chemotherapy regimen (i.e. addition or removal of at least one of the initial antineoplastic drugs) if this change occurred after the first 3 months of the study, or

(ii) at least 3 months and until the regimen ongoing at the 3-month time point is changed (change being defined as addition or removal of at least one of the antineoplastic drugs of the previous regimen) if the change in the initial chemotherapy regimen occurred within the first 3 months of the study and the patient continued on chemotherapy, or

(iii) until decision was made to stop definitely chemotherapy, if it occurred within the first three months of the study,

whichever came first.

Randomized treatment was allocated to eligible patients through a centralized randomization system using an Interactive Voice Response System (IVRS). In order to balance treatment groups with regard to prognostic factors and geographical region, a dynamic allocation was used taking into account three factors: the localization of the primary site of tumor (lung, pancreas, stomach, colon/rectum, bladder or ovary), the stage of the cancer (metastatic versus locally-advanced) and the geographical region (North America, South America, Western Europe, Eastern Europe, Asia and rest of the world). The randomization call occurred on the first day of the chemotherapy or the day after at the latest and as close as possible prior to the first IP injection.

During the study treatment period, visits were performed monthly±1 week (corresponding to scheduled chemotherapy visits). Signs and symptoms of VTE, bleeding, adverse events, specific concomitant medications (including chemotherapy) and compliance were assessed. Blood samples are drawn for laboratory tests and in all patients from selected centers for pharmacokinetic purposes.

At any time during the course of the study, if a patient experienced signs or symptoms evocative of VTE, unscheduled diagnostic test(s) were performed to confirm or rule out the presence of VTE. In addition, if a PE was discovered incidentally on a lung imaging test for tumor evaluation, a package was sent to the Blinded Adjudication Committee for review. All bleeding events and deaths reported up to the follow-up visit were adjudicated. In all these cases, the adjudication package consisted of relevant documentation such as all relevant films (venography or ultrasound for DVT, ventilation/perfusion lung scan, pulmonary angiogram or spiral CT lung scan for PE or any other imaging test leading to incidental VTE discovery) or autopsy report if available.

An end of treatment visit was done within 5 days after last study drug administration.

A follow-up visit was scheduled one month±1 week after the end of treatment visit. During this visit, information regarding adverse events (including bleedings and VTE) was collected.

In addition, survival status (alive, dead, or lost to follow up) was collected for all patients either one year after randomization or at the end of the study (i.e. 7 months following randomization of the last patient at the latest), whichever came first.

The duration of study participation per patient was variable and depended on the duration of chemotherapy. For a given patient, the duration of study period is the duration of study treatment followed by a one month follow-up period after the end of treatment visit. In addition, patients were screened within 3 weeks prior to the start of chemotherapy.

In any case, the study end date was at the latest seven months (6 months treatment period and one month follow-up) following the randomization of the last patient.

3) Selection of Patients

3.1: Inclusion Criteria

Patients eligible for inclusion in the study were cancer patients:
with metastatic or locally advanced solid tumor of the lung, pancreas, stomach, colon/rectum, bladder or ovary,
planned to start a (new) course of chemotherapy with a minimum intent of 3 months therapy.

Inclusion in the study also required that the patients had signed informed consent.

Chemotherapy is herein defined as any conventional cytotoxic treatment. Biological agents used alone were not considered as chemotherapy, but could be associated with cytotoxic agents.

3.2: Exclusion Criteria

3.2.2: Exclusion Criteria Related to Study Methodology

1. Legal lower age limitations (country specific).

2. Life expectancy less than 3 months.

3. ECOG (Eastern Cooperative Oncology Group) performance status of 3 or 4 (as published by Oken, M. M. et al. in Am. J. Clin. Oncol., 1982, vol. 5, pp. 649-655).

4. Calculated creatinine clearance<30 mL/min according to Cockroft and Gault formula (Nephron, 1976, vol. 16, pp. 31-41), i.e. severe renal impairment.

5. Any major surgery (i.e. open surgery lasting more than 45 minutes from opening to closure) within the last 4 weeks or planned during the study treatment period.

6. Contra-indications to anticoagulation:
active or recent (<3 months) significant bleeding, including gastrointestinal bleeding or peptic ulcer;
history of bleeding disorder (congenital, acquired or unexplained repeated bleeding episodes);
uncontrolled arterial hypertension (systolic blood pressure>180 mm Hg or diastolic blood pressure>110 mm Hg);
hemorrhagic stroke or recent (in the last 3 months) brain, spinal or ophthalmic surgery;
known cerebral hemorrhagic lesion;
primary or metastatic tumor which is at high bleeding-risk according to investigator's judgment;
known structural damage or other pathologic process involving the central nervous system (brain metastases, vascular malformation . . . );
thrombocytopenia (platelet count<100×10⁹/l);
activated partial thromboplastin time (aPTT)>1.5 ULN or International Normalized ratio (INR)>1.5.

7. Any treatment with other anti-thrombotic agents within 2 weeks prior to randomization or planned during the study treatment period, such as:
parenteral anticoagulants (UFH, LMWH: enoxaparin, dalteparin, nadroparin . . . , or other agents such as fondaparinux, bivalirudin, hirudin);
oral anticoagulants (Vitamin K antagonists);
anti-GPIIb/IIIa (eptifibatide, tirofiban, abciximab);
thrombolytic agents.

Notes:
Chronic treatment with anti-platelet agents such as low dose of aspirin (up to 325 mg/day) or clopidogrel or ticlopidine in patients with coronary artery disease was allowed.
To maintain patency of central venous catheter, saline flushing solutions were highly recommended. However, if it is local practice, flushing solutions with small amounts of heparin (max 500 units) was allowed.
Should an occlusion of the CVC occur, it was advised an attempt to restore the CVC patency by infusion of urokinase 5000-10000 units (could be repeated up to a dose of 50 000 units) or alteplase 2 mg.

8. Subject who requires a systematic venous thromboprophylaxis with anticoagulant or a curative anti-coagulant or thrombolytic treatment.

9. Pelvic venous obstruction or superior vena cava syndrome.

10. Subject unlikely to comply with protocol, e.g. uncooperative attitude, inability to return for follow-up visits, inability to receive daily injection (self-injection or by relative of patient or by health care professional) and unlikelihood of completing the study.

11. Treatment with any investigational product or investigational device in the last 30 days or 5 half lives (whichever is longer, if relevant) prior to randomization.

12. Any previous exposure to AVE5026 (e.g. participation in any previous AVE5026 clinical trial).

Note: A patient could not be randomized in the study more than once.

3.2.3: Exclusion Criteria Related to AVE5026

13. History of heparin-induced thrombocytopenia.

14. Known hypersensitivity to UFH or LMWH.

15. Pregnant or nursing woman or women of childbearing potential not protected by highly effective contraceptive method of birth control as defined for contraception in the Informed Consent Form and/or in a local protocol addendum for the duration of the study and/or who are unwilling or unable to be tested for pregnancy.

4) Treatments

Patients were allocated to one of the two study treatments: AVE5026 or placebo of AVE5026.

The AVE5026 syringe contained 20 mg of AVE5026 in a 0.5 mL pre-filled syringe containing 0.4 mL of a sterile, isotonic solution with sodium chloride 0.9% and water for injection corresponding to a concentration of 50 mg/mL.

The matching placebo syringe was strictly identical in appearance, containing the same volume but without active component.

AVE5026 or its placebo was administered subcutaneously.

Study medication (AVE5026 or its placebo) started as close as possible after the randomization, which should take place as close as possible to the start of chemotherapy. The first injection was done at site under direct supervision. The investigator decided whether injections at home could be performed by the patient (self-injection) or by a relative, or whether it should be supported by a health care professional.

Study medication was administered once daily s.c. at approximately 24 hours apart. The time of the day was upon investigator's or patient's preference. However it was recommended to keep the same timing during the study.

5) Assessment of Investigational Product

5.1: Efficacy

The primary efficacy criterion was the time-to-first occurrence of any component of the composite endpoint of the following documented outcome results, confirmed by the CIAC (composed of thrombosis and bleeding experts, blinded to study medication assignment), from randomization up to 3 calendar days after last IP injection:
Any symptomatic DVT of the lower limbs,
Any symptomatic DVT of the upper limbs (including CVC-related thrombosis),
Any non-fatal PE,
VTE-related deaths (including fatal PE and unexplained deaths).

The VTE diagnosis needed to be confirmed or ruled out by objective investigations, described as follows.
DVT of the lower limbs: the clinical diagnosis must be confirmed by compression ultrasound (CUS) or venography performed within 72 hours after the clinical suspicion. DVT was confirmed if the CUS was abnormal or if there was an intraluminal filling defect on the venography.
DVT of the upper limbs: thrombosis of the central line was not considered a priori as a suspicion of DVT unless a patient presented symptoms such as arm swelling, erythema, pain, distal paresthesias, neck swelling, headache, and congestion of subcutaneous collateral veins. In any case, the clinical diagnosis must be confirmed by ultrasound (US) or venography performed within 72 hours after the clinical suspicion. DVT was confirmed if the US is abnormal or if there was an intraluminal filling defect on the venography.
Pulmonary embolism: the clinical diagnosis must be confirmed by ventilation/perfusion lung scan, pulmonary angiogram or spiral Computer Tomography (CT) lung scan within 72 hours after the clinical suspicion. PE was confirmed in case of:
   intraluminal filling defect in (sub)segmental or more proximal branches on a spiral CT scan, or
   intraluminal filling defect on the pulmonary angiogram, or
   a perfusion defect of at least 75% of a segment with a local normal ventilation result (high-probability) on ventilation/perfusion lung scan, or
   an inconclusive spiral CT, pulmonary angiography or lung scintigraphy with demonstration of DVT in the lower extremities by compression ultrasound or venography, or
   a fatal PE based on autopsy.

The patient population used in the analysis of the primary efficacy endpoint (i.e. primary efficacy population) was the Intent-To-Treat (ITT) population, which included all randomized patients. Patients were analyzed in the treatment group to which they were allocated by the IVRS (i.e. “as randomized” regardless of treatment actually received).

5.2: Safety

The safety analysis period was defined as the period from the first IP injection up to the last IP injection plus 3 calendar days (called “on-treatment period”). The safety population was defined as all randomized patients exposed to the study medication, regardless of the amount of treatment administered.

Clinical safety was assessed by bleedings (major bleeding and clinically relevant non-major bleeding), vital signs, transfusions requirement, hemoglobin, platelet count, liver and renal laboratory data, (S)AEs and deaths (classified as VTE-related death, fatal bleeding or other by a blinded Adjudication Committee) up to 3 calendar days after last IP injection and up to the follow-up visit.

The definition of major bleeding was in agreement with the International Society on Thrombosis and Haemostasis (ISTH) recommendation for clinical investigations of antihemostatic products in non-surgical patients (J. Thromb. Haemost., 2005, vol. 3, pp. 692-4), namely:
Fatal bleeding, and/or
Symptomatic bleeding in a critical area or organ, such as intracranial, intraspinal, intraocular, retroperitoneal, intraarticular or pericardial, or intramuscular with compartment syndrome, and/or
Bleeding causing a fall in hemoglobin level of 2 g/dL (1.24 mmol/L) or more, or leading to transfusion of two or more units of whole blood or red cells

Overt bleedings requiring a medical intervention and not meeting any of the criteria for major bleeding were classified as “clinically relevant non-major”. Bleedings events others than major or clinically relevant non-major bleedings were classified as non-clinically relevant bleedings.

6) Results Concerning Study Patients and Treatment

A total of 3212 patients were randomized in the study, 1608 in the semuloparin group and 1604 in the placebo group. Amongst the randomized patients, 68.2% had a metastatic cancer and 31.8% had a locally advanced cancer.

The median duration of study treatment was around 3.5 months in the two groups, with most of patients receiving 3 to 6 months of study treatment: 48.8% in the semuloparin group and 48.3% in the placebo group.

7) Efficacy Results

The primary analysis of the primary endpoint consisted of the comparison of the two treatment groups (AVE5026 and placebo) using the two-sample test of Gray for comparing Cumulative Incidence Functions (CIFs), at a significant level of 0.05 (2-sided). An estimation of the treatment effect (hazard ratio and 95% CIs) was provided using Fine and Gray regression model for CIFs.

CIFs were estimated separately for the two treatment groups with Prentice non-parametric estimator using a model of cause-specific hazards; corresponding 95% 2-sided CIs were computed by Keiding and Andersen formula with variance computed using the delta method.

The event rates of the primary efficacy endpoint (any VTE) and its components occurring during the efficacy analysis period were summarized by treatment group.

Table 1 describes the incidences of the primary efficacy endpoint of SAVE-ONCO study by treatment groups, while FIG. 1 represents the corresponding survival curves. Table 2 describes the results for each component of the primary efficacy endpoint and table 3 describes the key subgroups analyses by cancer stage and location of primary tumor.

TABLE 1
Any VTE or VTE-related death during the efficacy
analysis period (time to first VTE event) - Primary
efficacy analysis - Intent-To-Treat population

| | AVE5026 | Placebo |
|---|---|---|
| | (N = 1608) | (N = 1604) |
| Any VTE or VTE-related death | 20 (1.2%) | 55 (3.4%) |
| [n (%)] | | |
| Comparison versus placebo: | | |
| Hazard ratio (95% CI) | | 0.36 (0.21 to 0.60) |
| Gray test p-value | | <0.0001 |
| Number of competing events * | 78 (4.9%) | 74 (4.6%) |
| [n (%)] | | |

N: number of patients in the primary efficacy population
n: number of patients showing a given event
* Competing events considered deaths from cause other than VTE occurring during the efficacy analysis period.

TABLE 2
Components of the primary efficacy endpoint

| | | | HR |
|---|---|---|---|
| | AVE5026 | Placebo | (95% mid- |
| | n/N (%) | n/N (%) | p CI) |
| Any VTE or VTE- | | | |
| related death | | | |
| n/N (%) | 20/1608 (1.2%) | 55/1604 (3.4%) | 0.36 |
| 95% mid-p CI | (0.8 to 1.9) | (2.6 to 4.4) | (0.21 to 0.60) |
| Symptomatic DVT | | | |
| n/N (%) | 11/1608 (0.7%) | 34/1604 (2.1%) | 0.32 |
| 95% mid-p CI | (0.4 to 1.2) | (1.5 to 2.9) | (0.15 to 0.62) |
| Symptomatic DVT | | | |
| of the upper limbs | | | |
| n/N (%) | 3/1608 (0.2%) | 9/1604 (0.6%) | 0.33 |
| 95% mid-p CI | (0.0 to 0.5) | (0.3 to 1.0) | (0.07 to 1.18) |
| Symptomatic DVT | | | |
| of the lower limbs | | | |
| n/N (%) | 8/1608 (0.5%) | 25/1604 (1.6%) | 0.32 |
| 95% mid-p CI | (0.2 to 0.9) | (1.0 to 2.3) | (0.13 to 0.69) |
| Proximal DVT of | | | |
| the lower limbs | | | |
| n/N (%) | 4/1608 (0.2%) | 19/1604 (1.2%) | 0.21 |
| 95% mid-p CI | (0.1 to 0.6) | (0.7 to 1.8) | (0.06 to 0.58) |
| Distal DVT of | | | |
| the lower limbs | | | |
| n/N (%) | 4/1608 (0.2%) | 12/1604 (0.7%) | 0.33 |
| 95% mid-p CI | (0.1 to 0.6) | (0.4 to 1.3) | (0.09 to 0.99) |
| PE | | | |
| n/N (%) | 10/1608 (0.6%) | 24/1604 (1.5%) | 0.41 |
| 95% mid-p CI | (0.3 to 1.1) | (1.0 to 2.2) | (0.20 to 0.86) |
| Non-fatal PE | | | |
| n/N (%) | 3/1608 (0.2%) | 15/1604 (0.9%) | 0.20 |
| 95% mid-p CI | (0.0 to 0.5) | (0.5 to 1.5) | (0.05 to 0.63) |
| Any VTE- | | | |
| related death | | | |
| n/N (%) | 7/1608 (0.4%) | 9/1604 (0.6%) | 0.77 |
| 95% mid-p CI | (0.2 to 0.9) | (0.3 to 1.0) | (0.27 to 2.13) |

TABLE 3
Any VTE or VTE-related death during the efficacy analysis period (time
to first VTE event) - Subgroups analyses - Intent-To-Treat population

| | AVE5026 | Placebo | Hazard ratio | Interaction |
|---|---|---|---|---|
| | n/N (%) | n/N (%) | (95% CI) | p-value |
| Stage of cancer: | | | | |
| metastatic | 16/1097 (1.5%) | 38/1095 (3.5%) | 0.42 (0.23 to 0.75) | 0.3236 |
| locally advanced | 4/511 (0.8%) | 17/509 (3.3%) | 0.23 (0.08 to 0.68) | |
| Location site of | | | | |
| primary tumor: | | | | |
| Lung | 9/951 (1.5%) | 25/589 (4.2%) | 0.36 (0.17 to 0.77) | 0.7994 |
| Pancreas | 3/126 (2.4%) | 14/128 (10.9%) | 0.22 (0.06 to 0.76) | |
| Stomach | 1/204 (0.5%) | 4/207 (1.9%) | 0.25 (0.03 to 2.20) | |
| Colon/rectum | 5/464 (1.1%) | 9/461 (2.0%) | 0.54 (0.18 to 1.60) | |
| Bladder | 1/32 (3.1%) | 3/31 (9.7%) | 0.30 (0.03 to 2.95) | |
| Ovary | 1/191 (0.5%) | 0/188 | NA | |

NA: not applicable

These tables demonstrate a significant superiority of semuloparin versus placebo on the composite primary endpoint (table 1), with a consistent treatment effect over the individual components of the primary efficacy endpoint, DVT and PE (table 2).

As apparent from tables 1 and 2, risk reduction compared to placebo was 64% for any VTE or VTE-related death (primary endpoint), 68% for DVT (67% for DVT of the upper limbs and 68% for DVT of the lower limbs) and 59% for PE.

FIG. 1 shows that the two curves for the events of interest in the semuloparin and placebo group diverge almost immediately after initiation of therapy, with a treatment effect consistent throughout the treatment period.

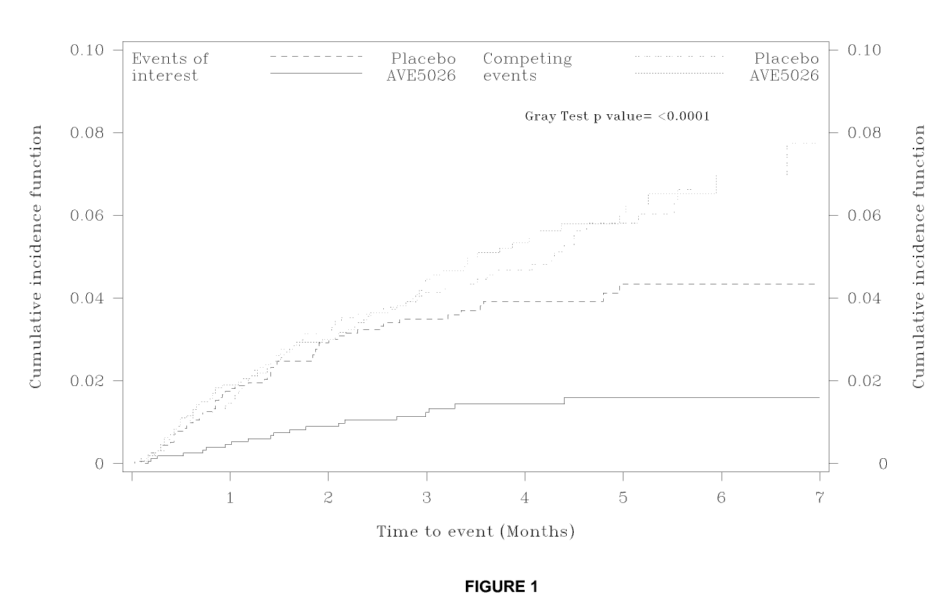

The additional analyses amongst subgroups of the Intent-To-Treat population (table 3) show that no heterogeneity of effect was detected across stage of cancer (metastatic or locally advanced) (interaction p-value=0.3236) and across location site of primary tumor (lung, pancreas, stomach, colon/rectum, bladder or ovary) (interaction p-value=0.7994).

8) Safety Results

CIFs were estimated separately for the two treatment groups together with 95% 2-sided CIs. An estimation of the treatment effect (hazard ratio and 95% CIs) was given using Fine and Gray regression model for CIFs.

Table 4 describes the incidence of any treatment emergent clinically relevant bleeding, major bleeding and clinically relevant non-major bleeding in the safety population of the SAVE-ONCO study, while table 5 describes the incidence of clinically relevant bleedings and of major bleedings by location site of primary tumor.

TABLE 4
Number of patients with treatment emergent clinically relevant bleedings,
major bleedings and non-major bleedings - Safety population

| | | | OR |
|---|---|---|---|
| | AVE5026 | Placebo | (95% mid- |
| | (N = 1589) | (N = 1583) | p CI) |
| Any clinically | | | |
| relevant bleeding: | | | |
| n (%) | 45 (2.8%) | 32 (2.0%) | 1.41 |
| 95% mid-p CI | (2.1 to 3.7) | (1.4 to 2.8) | (0.89 to 2.25) |
| Any major | | | |
| bleeding: | | | |
| n (%) | 19 (1.2%) | 18 (1.1%) | 1.05 |
| 95% mid-p CI | (0.7 to 1.8) | (0.7 to 1.8) | (0.55 to 2.04) |
| Any clinically | | | |
| relevant non-major | | | |
| bleeding: | | | |
| n (%) | 26 (1.6%) | 14 (0.9%) | 1.86 |
| 95% mid-p CI | (1.1 to 2.4) | (0.5 to 1.4) | (0.98 to 3.68) |

N: number of patients in the safety population
n: number of patients with bleedings

This table shows that the incidence of clinically relevant bleedings, including clinically relevant non-major bleedings, is slightly higher in the semuloparin group vs. placebo, and is consistent with what is expected for an antithrombotic product. However, the incidence of major bleedings is similar between the two studied groups (OR=1.05, 95% mid-p CI [0.55 to 2.04]).

TABLE 5
Number of patients with clinically relevant or major bleedings -
Subgroup analysis by cancer type - Safety population

| Location site | AVE5026 | Placebo | Hazard ratio | Interaction |
|---|---|---|---|---|
| of primary tumor | n/N (%) | n/N (%) | (95% CI) | p-value |
| Clinically relevant | | | | |
| bleedings: | | | | |
| Lung | 15/586 (2.6%) | 11/579 (1.9%) | 1.39 (0.64 to 3.01) | 0.6899 |
| Pancreas | 3/121 (2.5%) | 5/127 (3.9%) | 0.65 (0.16 to 2.65) | |
| Stomach | 5/204 (2.5%) | 2/206 (1.0%) | 2.46 (0.48 to 12.59) | |
| Colon/rectum | 16/458 (3.5%) | 11/455 (2.4%) | 1.42 (0.66 to 3.03) | |
| Bladder | 2/32 (6.3%) | 2/30 (6.7%) | 0.94 (0.13 to 6.62) | |
| Ovary | 4/188 (2.1%) | 1/186 (0.5%) | 3.94 (0.45 to 34.52) | |
| Major bleedings: | | | | |
| Lung | 8/586 (1.4%) | 6/579 (1.0%) | 1.38 (0.48 to 3.95) | NA |
| Pancreas | 2/121 (1.7%) | 5/127 (3.9%) | 0.40 (0.08 to 2.03) | |
| Stomach | 4/204 (2.0%) | 1/206 (0.5%) | 3.95 (0.44 to 35.03) | |
| Colon/rectum | 3/458 (0.7%) | 6/455 (1.3%) | 0.48 (0.12 to 1.91) | |
| Bladder | 0/32 | 0/30 | NA | |
| Ovary | 2/188 (1.1%) | 0/186 | NA | |

NA: not applicable

The results by subgroups according to table 5 are useful for the assessment of the benefit-to-risk ratio of semuloparin treatment as detailed hereafter.

9) Clinical Net Benefit

The SAVE-ONCO study has demonstrated the benefit of thromboprophylaxis with semuloparin in patients receiving chemotherapy without increase in the incidence of major bleeding.

This study has demonstrated that semuloparin exhibits a favorable benefit-to-risk ratio for all following efficacy and safety outcomes:
any VTE or VTE-related death versus any major bleeding;
any VTE or VTE-related death versus any clinically relevant bleeding;
any PE versus any major bleeding;
any PE versus any fatal bleeding; and
any VTE-related death versus any fatal bleeding.

The results obtained show that compared to placebo, for every 1000 patients treated, semuloparin 20 mg q.d. prevents 22 VTE or VTE-related deaths and causes <1 more major bleeding.

10) Benefit-to-Risk Assessment According to VTE Risk

Recent oncology guidelines emphasize the need for randomized studies with VTE risk assessment in the patients.

Standard (i.e. applicable to the general population regardless of malignancy) VTE risk factors are selected from: presence of a CVL (Central Venous Line), obesity, age greater or equal to 75 years, history of VTE (prior DVT or PE), chronic respiratory failure, chronic heart failure, hormonal therapy and venous insufficiency (e.g. presence of varicose veins).

As regards cancer specific VTE risk factors, baseline VTE risk was assessed by a score specifically developed and validated in chemotherapy-treated cancer patients (Khorana A. A. et al., Blood, 2008, vol. 111, 4902-7). According to this predictive model a score of 2 was assigned to very high-risk cancer sites (pancreatic or gastric), a score of 1 was assigned to high-risk cancer sites (lung, ovarian or bladder cancer) and 1 is added to the score for each of the following parameters: platelet count≧350×10⁹/L, hemoglobin<10 g/dL and/or use of erythropoietin-stimulating agents, leukocyte count>11×10⁹/L, and Body Mass Index≧35 kg/m².

Tables 6 and 7 describe the baseline (i.e. at patients enrollment) characteristics of the SAVE-ONCO patient population according to standard risk factors for VTE (table 6) and to cancer-specific VTE risk score (table 7), while table 8 summarizes the number of patients with at least one overall additional VTE risk factor (whether cancer-specific or standard). It is apparent from these tables that approximately 80% of the patients had a cancer-specific VTE risk score≧1, and that more than 40% of the patients had ≧1 standard VTE risk factor in addition to the VTE risk posed by cancer and chemotherapy.

TABLE 6
Baseline characteristics of the SAVE-ONCO population
according to standard risk factors for VTE

| | AVE5026 | Placebo |
|---|---|---|
| | N = 1608 | N = 1604 |
| | n (%) | n (%) |
| Standard risk factors for VTE: | 685 (42.6%) | 672 (41.9%) |
| CVL | 316 (19.7%) | 302 (18.8%) |
| Obesity | 208 (12.9%) | 206 (12.8%) |
| Age ≧75 | 111 (6.9%) | 106 (6.6%) |
| Chronic respiratory failure | 78 (4.9%) | 91 (5.7%) |
| Venous insufficiency/varicose veins | 92 (5.7%) | 76 (4.7%) |
| Chronic heart failure | 56 (3.5%) | 57 (3.6%) |
| History of DVT | 26 (1.6%) | 32 (2.0%) |
| Use of hormonal therapy | 21 (1.3%) | 27 (1.7%) |
| History of PE | 6 (0.4%) | 5 (0.3%) |

N: number of patients in the primary efficacy population
n: number of patients with a given risk factor

TABLE 7
Baseline characteristics of the SAVE-ONCO population
according to cancer-specific VTE risk score

| | AVE5026 | Placebo | Total |
|---|---|---|---|
| | N = 1608 | N = 1604 | N = 3212 |
| VTE risk score | n (%) | n (%) | n (%) |
| 0 (low risk) | 313 (19.8%) | 301 (19.0%) | 614 (19.4%) |
| 1-2 (moderate risk) | 995 (63.0%) | 1003 (63.3%) | 1998 (63.2%) |
| ≧3 (high risk) | 271 (17.2%) | 279 (17.6%) | 550 (17.4%) |

N.B.: The sum of patients in the various sub-groups is less than 1608 and 1604, respectively, due to missing data for some patients.

TABLE 8
Patients with at least one overall additional VTE risk factor

| | AVE5026 | Placebo |
|---|---|---|
| At least one overall | N = 1608 | N = 1604 |
| additional VTE risk factor | n (%) | n (%) |
| Yes | 1072 (67%) | 1084 (68%) |
| No | 536 (33%) | 520 (32%) |

Efficacy results according to the primary endpoint (VTE or VTE-related death) show that the incidence of VTE increased proportionally to the number of standard VTE risk factors and to the cancer-specific VTE risk score, and that the treatment effect using semuloparin was consistent across various levels of VTE risk, whether standard or cancer-specific (see tables 9 and 10).

TABLE 9
Efficacy and safety (bleedings) outcomes by number of standard VTE risk factors

| | AVE5026 | Placebo | HR | Interaction |
|---|---|---|---|---|
| | n/N (%) | n/N (%) | (95% CI) | p-value |
| VTE or VTE-related death: | | | | |
| None | 8/895 (0.9%) | 23/932 (2.5%) | 0.39 (0.18-0.84) | |
| 1 or 2 | 9/635 (1.4%) | 27/632 (4.3%) | 0.32 (0.15-0.68) | 0.8358 |
| ≧3 | 2/33 (6.1%) | 5/40 (12.5%) | 0.56 (0.11-2.93) | |
| Clinically relevant bleedings: | | | | |
| None | 23/914 (2.5%) | 19/923 (2.1%) | 1.20 (0.65-2.21) | |
| 1 or 2 | 19/643 (3.0%) | 11/620 (1.8%) | 1.68 (0.80-3.51) | 0.7161 |
| ≧3 | 3/32 (9.4%) | 2/40 (5.0%) | 1.79 (0.33-9.79) | |
| Major bleedings: | | | | |
| None | 9/914 (1.0%) | 8/923 (0.9%) | 1.11 -0.43-2.87) | |
| 1 or 2 | 8/643 (1.2%) | 8/620 (1.3%) | 0.97 (0.37-2.57) | 0.9391 |
| ≧3 | 2/32 (6.3%) | 2/40 (5.0%) | 1.16 (0.18-7.61) | |

n/N: number of patients in the selected category showing a given effect compared to the total number of patients in said category.

TABLE 10
Efficacy and safety (clinically relevant and major bleedings)
outcomes by baseline VTE risk score (cancer-specific)

| | AVE5026 | Placebo | HR | Interaction |
|---|---|---|---|---|
| | n/N (%) | n/N (%) | (95% CI) | p-value |
| VTE or VTE-related death: | | | | |
| VTE risk score 0 | 3/313 (1.0%) | 4/301 (1.3%) | 0.71 (0.16-3.15) | 0.6048 |
| VTE risk score 1-2 | 13/995 (1.3%) | 35/1003 (3.5%) | 0.37 (0.20-0.70) | |
| VTE risk score ≧3 | 4/271 (1.5%) | 15/279 (5.4%) | 0.27 (0.09-0.82) | |
| Clinically relevant bleedings: | | | | |
| VTE risk score 0 | 8/310 (2.6%) | 6/297 (2.0%) | 1.34 (0.48-3.78) | 0.9409 |
| VTE risk score 1-2 | 28/987 (2.8%) | 19/988 (1.9%) | 1.48 (0.83-2.65) | |
| VTE risk score ≧3 | 9/264 (3.4%) | 7/277 (2.5%) | 1.26 (0.47-3.34) | |
| Major bleedings: | | | | |
| VTE risk score 0 | 2/310 (0.6%) | 2/297 (0.7%) | 1.13 (0.18-6.99) | 0.9845 |
| VTE risk score 1-2 | 12/987 (1.2%) | 11/988 (1.1%) | 1.09 (0.48-2.47) | |
| VTE risk score ≧3 | 5/264 (1.9%) | 5/277 (1.8%) | 1.01 (0.30-3.48) | |

The results in table 10 show that no pattern of semuloparin-related increased major bleeding risk was detected in patients at increased risk of VTE.

It can be concluded from these results that thromboprophylaxis with semuloparin was consistently associated with a favorable benefit-to-risk profile across various levels of VTE risk, with the greatest benefit-to-risk profile in moderate to high risk patients (VTE risk score ranging from 1 to 3 or above).

Benefit-to-risk analysis in patients with at least one overall additional VTE risk factor (whether standard or cancer-specific) shows that thromboprophylaxis with semuloparin was associated with a favorable benefit-to-risk profile in said patient population (see table 11 below).

TABLE 11
Efficacy and safety outcomes in patients with at least one overall
additional VTE risk factor (whether standard or cancer-specific).

| | AVE5026 | Placebo | HR |
|---|---|---|---|
| | n/N (%) | n/N (%) | (95% CI) |
| VTE or VTE- | 16/1072 (1.5%) | 41/1084 (3.8%) | 0.39 (0.22-0.69) |
| related death | | | |
| Clinically | 33/1055 (3.1%) | 24/1067 (2.2%) | 1.37 (0.81-2.31) |
| relevant bleedings | | | |
| Major bleedings | 12/1055 (1.1%) | 17/1067 (1.6%) | 0.70 (0.33-1.46) |

The beneficial effect of semuloparin was therefore observed across tumor location, stage of disease, and number of VTE risk factors. The highest benefit of semuloparin thromboprophylaxis was observed in patients at increased risk of VTE, namely:
patients with pancreatic cancer (HR=0.22, 95% CI=0.06-0.76),
patients with lung cancer (HR=0.36, 95% CI=0.17-0.77), and
patients with a least one VTE risk factor in addition to the VTE risk posed by cancer and chemotherapy (HR=0.39, 95% CI=0.22-0.69).

11) Efficacy and Safety in Patients with Pancreatic or Lung Cancer or with a Cancer-Specific VTE Risk Score≧3

Additional analyses in specific subgroups of patients further show that the highest absolute benefit and optimum benefit-to-risk ratio are observed in patients with the highest risk of VTE, namely patients with pancreatic cancer, lung cancer or with a cancer-specific VTE risk score equal to or greater than 3.

Indeed, any VTE or VTE-related death occurred in 14 (10.9%) patients in the placebo group versus 3 (2.4%) patients in the group treated with semuloparin amongst patients with pancreatic cancer (table 3); in 25 (4.2%) patients in the placebo group versus 9 (1.5%) patients in the group treated with semuloparin amongst patients with lung cancer (table 3); and in 15 (5.4%) patients in the placebo group versus 4 (1.5%) patients in the group treated with semuloparin amongst patients with a VTE risk score (cancer-specific)≧3 (table 10). In addition, no pattern of increased bleeding in the semuloparin arm was observed in patients at increased risk of VTE due to the cancer type (lung or pancreas, see table 5) or due to the presence of additional VTE risk factors (table 10).

Efficacy and safety results in the patients with pancreatic or lung cancer or with a cancer-specific VTE risk score≧3 are described below (tables 12 to 16).

TABLE 12
Any VTE or VTE-related death during the efficacy analysis
period - Patients with pancreas cancer or lung cancer
or VTE risk score ≧3 - Intent-To-Treat population

| | AVE5026 | Placebo |
|---|---|---|
| | (N = 837) | (N = 837) |
| VTE or VTE- | 12 (1.4%) | 43 (5.1%) |
| related death [n (%)] | | |
| Comparison versus placebo: | | |
| Hazard ratio (95% CI) | | 0.28 (0.15 to 0.53) |
| Gray test p-value | | <0.0001 |
| Number of | 53 (6.3%) | 53 (6.3%) |
| competing events * [n (%)] | | |

N: number of patients in the selected population
n: number of patients showing a given event
* Competing events considered deaths from cause other than VTE-related death occurring during the efficacy analysis period.

TABLE 13
Any VTE or VTE-related death during the efficacy analysis
period - Components of the primary efficacy endpoint -
Patients with pancreas cancer or lung cancer or VTE
risk score ≧3 - Intent-To-Treat population

| | | | OR |
|---|---|---|---|
| | AVE5026 | Placebo | (95% mid- |
| | n/N (%) | n/N (%) | p CI) |
| Any VTE or VTE- | | | |
| related death | | | |
| n/N (%) | 12/837 (1.4%) | 43/837 (5.1%) | 0.27 |
| 95% mid-p CI | (0.8 to 2.4) | (3.8 to 6.8) | (0.14 to 0.50) |
| Symptomatic DVT | | | |
| n/N (%) | 5/837 (0.6%) | 24/837 (2.9%) | 0.20 |
| 95% mid-p CI | (0.2 to 1.3) | (1.9 to 4.2) | (0.07 to 0.51) |
| Symptomatic DVT of | | | |
| the upper limbs | | | |
| n/N (%) | 1/837 (0.1%) | 6/837 (0.7%) | 0.17 |
| 95% mid-p CI | (0.0 to 0.6) | (0.3 to 1.5) | (0.01 to 1.13) |
| Symptomatic DVT of | | | |
| the lower limbs | | | |
| n/N (%) | 4/837 (0.5%) | 18/837 (2.2%) | 0.22 |
| 95% mid-p CI | (0.2 to 1.1) | (1.3 to 3.3) | (0.06 to 0.61) |
| Proximal DVT of | | | |
| the lower limbs | | | |
| n/N (%) | 1/837 (0.1%) | 12/837 (1.4%) | 0.08 |
| 95% mid-p CI | (0.0 to 0.6) | (0.8 to 2.4) | (0.00 to 0.48) |
| Distal DVT of | | | |
| the lower limbs | | | |
| n/N (%) | 3/837 (0.4%) | 10/837 (1.2%) | 0.30 |
| 95% mid-p CI | (0.1 to 1.0) | (0.6 to 2.1) | (0.07 to 1.03) |
| PE | | | |
| n/N (%) | 8/837 (1.0%) | 22/837 (2.6%) | 0.36 |
| 95% mid-p CI | (0.4 to 1.8) | (1.7 to 3.9) | (0.15 to 0.79) |
| Non-fatal PE | | | |
| n/N (%) | 3/837 (0.4%) | 15/837 (1.8%) | 0.20 |
| 95% mid-p CI | (0.1 to 1.0) | (1.0 to 2.9) | (0.05 to 0.63) |
| Any VTE- | | | |
| related death | | | |
| n/N (%) | 5/837 (0.6%) | 7/837 (0.8%) | 0.71 |
| 95% mid-p CI | (0.2 to 1.3) | (0.4 to 1.6) | (0.21 to 2.31) |

Notes:
Patients with several events may be counted in several categories.
VTE-related death includes fatal PE and unexplained deaths.

TABLE 14
Number of patients with treatment-emergent clinically relevant
bleedings, and major bleedings - Patients with pancreas cancer
or lung cancer or VTE risk score ≧3 - Safety population

| | | | OR |
|---|---|---|---|
| | AVE5026 | Placebo | (95% mid- |
| | (N = 824) | (N = 825) | p CI) |
| Any clinically | | | |
| relevant bleeding: | | | |
| n (%) | 23 (2.8%) | 17 (2.1%) | 1.36 |
| 95% mid-p CI | (1.8 to 4.1) | (1.2 to 3.2) | (0.72 to 2.62) |
| Any major bleeding: | | | |
| n (%) | 13 (1.6%) | 11 (1.3%) | 1.19 |
| 95% mid-p CI | (0.9 to 2.6) | (0.7 to 2.3) | (0.52 to 2.73) |

N: number of patients in the safety population
n: number of patients with bleedings

The analysis of time to first event of any VTE or VTE-related death or major bleeding demonstrated the favorable benefit-risk of semuloparin versus placebo in the selected population of patients with pancreas cancer or lung cancer or VTE risk score≧3:25 [3.0%] events in the semuloparin group versus 54 [6.5%] events in the placebo group, hazard ratio: 0.47 [95% CI: 0.29-0.75] (see table 15).

TABLE 15
Any VTE or VTE-related death or major bleeding during the on-
treatment period - Patients with pancreas cancer or lung cancer
or VTE risk score ≧3 - Randomized and exposed population

| | AVE5026 | Placebo |
|---|---|---|
| | (N = 824) | (N = 825) |
| Any VTE or major bleedings | 25 (3.0%) | 54 (6.5%) |
| [n (%)] | | |
| Number of competing events * | 52 (6.3%) | 50 (6.1%) |
| [n (%)] | | |
| Comparison versus placebo: | | |
| Hazard ratio (95% CI) | | 0.47 (0.29 to 0.75) |

* Competing events considered deaths from cause other than VTE-related death or fatal bleedings occurring during the safety analysis period.

The benefit-to-risk ratio of semuloparin versus placebo in the selected population (patients with pancreas cancer or lung cancer or VTE risk score≧3), based on Absolute Risk Difference (ARD) and Number Needed to Treat (NNT) to prevent one VTE versus Number Needed to Harm (NNH) to cause one major bleeding, is shown in Table 16.

For every 1000 cancer patients in the target population treated (patients with pancreas cancer or lung cancer or VTE risk score≧3), semuloparin 20 mg q.d. would:
prevent 33 VTE or VTE-related deaths, and
cause<1 more major bleeding.

TABLE 16
Number needed to treat to prevent one VTE or VTE-related death
versus number needed to harm to cause one major bleeding

| | AVE5026 | Placebo | | |
|---|---|---|---|---|
| | n/N (%) | n/N (%) | ARD (%) | NNT-NNH |
| Any VTE or VTE-related death | 12/837 (1.4%) | 43/837 (5.1%) | −3.29% ^{a} | −30 ^{a} |
| Any major bleeding | 13/824 (1.6%) | 11/825 (1.3%) | 0.07% ^{b} | 1500 ^{b} |

^{a} Under assumption of consistent treatment effect (ie, HR = 0.36).
^{b} Under assumption of consistent treatment effect (ie, HR = 1.05).

What is claimed is:

1. A method for the prophylaxis of venous thromboembolism in a patient receiving chemotherapy for locally advanced or metastatic solid tumors, comprising administering to said patient an ultra-low molecular weight heparin with an average molecular weight of 2000 to 3000 Daltons, an anti-FXa activity of about 160 U/mg and an anti-IIa activity of about 2 U/mg.

2. The method according to claim 1, wherein said ultra-low molecular weight heparin is semuloparin or a pharmaceutically acceptable salt thereof.

3. The method according to claim 1, wherein said patient has lung, pancreas, stomach, colon, rectum, bladder or ovary cancer.

4. The method according to claim 2, wherein said patient has lung, pancreas, stomach, colon, rectum, bladder or ovary cancer.

5. The method according to claim 1, wherein said patient has lung, stomach, colon, rectum, bladder or ovary cancer.

6. The method according to claim 2, wherein said patient has lung, stomach, colon, rectum, bladder or ovary cancer.

7. The ultra-low molecular weight heparin for the use according to any one of claims 1 to 6, wherein said patient is at increased risk of VTE.

8. The method according to claim 1 or claim 2, wherein said patient is receiving chemotherapy for locally advanced or metastatic pancreatic or lung cancer, or for locally advanced or metastatic solid tumors and has a VTE risk score equal to or greater than 3.

9. The method according to claim 8, wherein said patient having a VTE risk score equal to or greater than 3 is selected from:
patients with stomach cancer who have, before initiating chemotherapy, one or more of the risk factors selected from: platelet count of 350×10⁹/L or more; hemoglobin less than 100 g/L or requiring red cell growth factors; leukocyte count more than 11×10⁹/L; body mass index of 35 kg/m² or more;
patients with bladder or ovarian cancer who have two or more of the risk factors defined above before initiating chemotherapy; and
patients with colon and/or rectum cancer who have three or more of the risk factors defined above before initiating chemotherapy.

10. The method according to claim 1 or claim 2, wherein said patient is an ambulatory patient.

11. The method according to claim 1 or claim 2, wherein said patient receives chemotherapy without concomitant treatment with thalidomide or lenalidomide.

12. A method for the prophylaxis of venous thromboembolism and venous thromboembolism-related death in a patient receiving chemotherapy for locally advanced or metastatic solid tumors, comprising administering to said patient an ultra-low molecular weight heparin with an average molecular weight of 2000 to 3000 Daltons, an anti-FXa activity of about 160 U/mg and an anti-IIa activity of about 2 U/mg.

13. The method according to claim 12, wherein said ultra-low molecular weight heparin is semuloparin or a pharmaceutically acceptable salt thereof.

14. The method according to claim 13, wherein said method involves a 64% risk reduction in the occurrence of venous thromboembolism and venous thromboembolism-related death, compared to placebo.

15. The method according to claim 1 or claim 2, wherein the venous thromboembolism is deep vein thrombosis.

16. The method according to claim 15, wherein said method involves a 68% risk reduction in the occurrence of deep vein thrombosis, compared to placebo.

17. The method according to claim 1 or claim 2, wherein the venous thromboembolism is deep vein thrombosis of the upper limbs.

18. The method according to claim 17, wherein said method involves a 67% risk reduction in the occurrence of deep vein thrombosis of the upper limbs, compared to placebo.

19. The method according to claim 1 or claim 2, wherein the venous thromboembolism is deep vein thrombosis of the lower limbs.

20. The method according to claim 19, wherein said method involves a 68% risk reduction in the occurrence of deep vein thrombosis of the lower limbs, compared to placebo.

21. The method according to claim 1 or claim 2, wherein the venous thromboembolism is pulmonary embolism.

22. The method according to claim 21, wherein said method involves a 59% risk reduction in the occurrence of pulmonary embolism, compared to placebo.

23. The method according to claim 2, wherein the efficacy and safety of the method are proven by a phase III clinical trial in cancer patients.

24. The method according to claim 2, wherein said ultra-low molecular weight heparin is administered at a 20 mg daily dose.

25. The method according to claim 2, wherein said ultra-low molecular weight heparin is administered for 3 months starting at the initiation of a course of chemotherapy.

26. An article of manufacture comprising:
a packaging material;
a compound chosen from an ULMWH with an average molecular weight of 2000 to 3000 Daltons, an anti-FXa activity of about 160 U/mg and an anti-FIIa activity of about 2 U/mg, in particular semuloparin or a pharmaceutically acceptable salt thereof; and
a label or package insert contained within said packaging material indicating that said compound is effective as an antithrombotic agent for the prophylaxis of venous thromboembolism (VTE) in cancer patients receiving chemotherapy for locally advanced or metastatic solid tumors.

27. A method of providing semuloparin or a pharmaceutically acceptable salt thereof, wherein said semuloparin or a pharmaceutically acceptable salt thereof is provided along with information describing that semuloparin or a pharmaceutically acceptable salt thereof is indicated in patients receiving chemotherapy for locally advanced or metastatic pancreatic or lung cancer or for locally advanced or metastatic solid tumors with a VTE risk score equal or greater than 3.

28. A method of promoting the use of semuloparin or a pharmaceutically acceptable salt thereof, the method comprising the step of conveying to a recipient at least one message chosen from:
semuloparin or a pharmaceutically acceptable salt thereof should be prescribed to a patient receiving chemotherapy for locally advanced or metastatic solid tumors;
semuloparin or a pharmaceutically acceptable salt thereof should be prescribed to a patient receiving chemotherapy for locally advanced or metastatic solid tumors of the lungs, pancreas, stomach, colon, rectum, bladder or ovaries;
semuloparin or a pharmaceutically acceptable salt thereof should be prescribed to a patient receiving chemotherapy for locally advanced or metastatic pancreatic cancer;
semuloparin or a pharmaceutically acceptable salt thereof should be prescribed to a patient receiving chemotherapy for locally advanced or metastatic lung cancer; and
semuloparin or a pharmaceutically acceptable salt thereof should be prescribed to a patient receiving chemotherapy for locally advanced or metastatic solid tumors with a VTE risk score equal or greater than 3.
